(19) United States
(12) Patent Application Publication
(10) Pub. No.: US 2010/0226972 A1
(43) Pub. Date: Sep. 9, 2010

(54) TRANSDERMAL PAIN GEL

(76) Inventor: George W. Lutz, Warren, NJ (US)

Correspondence Address: BARRY W. CHAPIN, ESQ.;CHAPIN INTELLECTUAL PROPERTY LAW, LLC, WESTBOROUGH OFFICE PARK, 1700 WEST PARK DRIVE, SUITE 280, WESTBOROUGH, MA, 01581, US

(21) Appl. No.: 12/718,417

(22) Filed: Mar. 5, 2010

Related U.S. Application Data
(60) Provisional application No. 61/158,161, filed on Mar. 6, 2009.

(51) Int. Cl. A61K 9/127; A61K 31/195

(52) U.S. Cl. 424/450; 514/567

(57) ABSTRACT

A transdermal gel including a complementary array of medicinal components has beneficial effects for pain relief in muscular and connective tissues. The medicinal components include active ingredients having a synergistic effect for permitting musculoskeletal movement by countering the symptoms of musculoskeletal pain and being non-narcotic for avoiding dependency, and are combined in a liposomal base with a wetting agent to form a gel consistency suitable for skin application. The transdermal gel allows topical application of greater quantities and concentrations of the active ingredients than could safely be obtained via conventional oral administration.

### CROSS REFERENCE TO RELATED APPLICATIONS

This patent application claims the benefit under 35 U.S.C. §119(e) of U.S. Provisional Patent Application No. 61/158,161 filed on Mar. 6, 2009, entitled, “TRANSDERMAL PAIN GEL,” the contents and teachings of which are hereby incorporated by reference in their entirety.

### BACKGROUND

Pain medication relieves unpleasant symptoms caused by physiological responses to injury or other perceived stimuli to actual or potential human tissue damage. Most often, pain medication and relief strives to block neurotransmitters which transmit pain stimuli to the brain that results in the uncomfortable pain sensations experienced by the afflicted person. Although human evolution has provided such pain recognition and the accompanying uncomfortable sensation as a means of self preservation, to inform the body of eminent possible tissue damage, once the initial pain sensation alerts the human subject, subsequent stimuli merely prolong the uncomfortable nature of the pain responsive neurotransmitters.

Neurotransmitters cause transmissions along nerve fibers comprised of neurons (nerve cells) that transmit minute electrical signals. Individual neurons transmit across a synapse defined by an intercell gap between individual neurons. The neurotransmitters travel in the synapse between individual neurons to transmit the pain stimuli, or signal. Thus, preventing neurotransmitters from crossing the synapse prevents transmission of pain stimuli. Typical pain medications deliver substances that interrupt the neurotransmitter path between neurons to avoid the painful sensation perceived by the human subject.

In particular, so-called glutamate transmitters are responsive at the spinal cord level and thus are often associated with chronic pain response. Glutamate is a neurotransmitter triggering spinal excitatory synaptic transmission and for generation and maintenance of spinal states of pain hypersensitivity via activation of glutamate receptors. The glutamate transporter system is the primary mechanism for the inactivation of synaptically released glutamate and the maintenance of glutamate homeostasis. Recent studies raise the suggestion that spinal glutamate transporter inhibition relieves pathological pain, suggesting that the spinal glutamate transporter might serve as a therapeutic target for treatment of pathological pain. In addition to its essential metabolic role, glutamate is a major mediator of excitatory signals in the central nervous system and is involved in many physiologic and pathologic processes, including chronic pain. Glutamate exerts its signaling role by acting on glutamate receptors, including N-methyl-D-aspartate (NMDA) receptors. These NMDA receptors are located on the pre- and post-synaptic membranes, as well as, at extra-synaptic sites. Glutamate concentration in the synapse therefore affects the extents of receptor stimulation and excitatory synaptic transmission. One cause of NMDA pain is that protracted activation of small primary afferent fibers can induce states of facilitated spinal sensory processing.

### SUMMARY

Pain medications provide relief by suppressing the nervous system of a patient, and typically operate by deactivating pain sensing mechanisms such that the pain impulses transported by the central nervous system (CNS) are not perceived by the patient. General anesthesia, often administered during surgery, incapacitates the entire CNS of a patient. Less invasive means, such as local anesthesia, are directed to selective immobilization or deactivation of certain areas, such as novocain administered during dental procedures. Often, pain medication disrupts the flow of chemical neurotransmitters in the synapse between individual neurons (nerve cells) that comprise the nerve fibers transmitting the pain impulses.

Configurations herein are based, in part, on the observation that conventional pain medication therapy may encounter difficulty focusing effective and appropriate dosage quantities to an afflicted area. Conventional approaches employ either oral or intravenous delivery mediums which disperse medication throughout the patients body, overbroadening the delivery area while delivering insufficient quantities to the afflicted area, and also possibly causing undesirable side effects. Further, narcotic based approaches, while providing adequate relief, may cause undesirable side effects such as sleepiness, loss of coordination, and dependency. It would be beneficial to provide a focused pain relief in a synergistic combination of components that delivers appropriate concentrations to an afflicted area to relieve pain, while reducing inflammation and allowing flexibility for permitting movement in the afflicted area. Accordingly, configurations herein provide a transdermal pain gel suitable for topical application and transdermal absorption directly to the afflicted area, such that transdermal absorption delivers medicinal components for suppressing NDMA receptors that otherwise transmit chronic pain stimuli triggering spinal cord sensitization. Substances that interfere and slow down or prevent the NMDA synapse transmission are so-called antagonists, and include ketamine and dextromethorphan. The symbiotic effect of medicinal components (components) for reduced inflammation and increased flexibility permit less restriction of movement while avoiding the side effects and general dexterous compromise of more general anesthetics.

Conventional pain medications suffer from the shortcoming that insufficient quantities of medication are directed to the afflicted areas. Oral administration of pain medication may require excessive doses in order to deliver an effective dose to the afflicted area, thus rendering oral ingestion insufficiently focused. Configurations disclosed herein substantially overcome the shortcomings of conventional pain relief by combining a complementary array of active ingredients, each of which provides a particular aspect of pain relief. The method combines active ingredients, detailed further below, in a liposomal base or other suitable medium, with a wetting agent to produce a creamlike gel suitable for application on the skin over a pain afflicted area. The suspension properties of the gel maintain the active ingredients in communication with the skin so as to provide transdermal absorption through the skin directly to the affected tissue areas, thus avoiding gastrointestinal ingestion and accompanying side effects of circulatory (bloodstream) delivery of the active ingredients.

A transdermal gel as disclosed further below includes a complementary array of active ingredients has beneficial effects for pain relief in muscular and connective tissues. The active ingredients are combined in a liposomal base with a wetting agent to form a gel or pastelike consistency suitable for skin application. The transdermal gel allows topical application of greater quantities and concentrations of the active ingredients than could safely be obtained via conventional oral administration.

Topical application on an external skin surface therefore delivers the medication to the afflicted area in a manner and quantity which would may be unsafe and/or impractical to deliver orally or intravenously to achieve a target dosing concentration. The disclosed transdermal gel includes medicinal components for achieving pain relief (via pain receptor blocking), an anti-inflamatory to prevent swelling, and a relaxant component having anti-spasmodic properties, thus achieving a synergistic combination and concentration of medicine focused on an afflicted area in a manner which is unachievable by oral or IV administration. The dosage quantity delivered via the transdermal delivery medium is typically of a prescriptive strength of one or more of the medicinal components, and is thus unavailable in an OTC manner.

### BRIEF DESCRIPTION OF THE DRAWINGS

The foregoing and other objects, features and advantages of the invention will be apparent from the following description of particular embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is a diagram of a neural synapse employing NMDA receptors responsive to the present invention; and

FIG. 2 is an anatomical cross section showing an epidermal region suitable for use with the transdermal gel of the present invention

### DETAILED DESCRIPTION

A major aspect of pain medication administration, and indeed an underpinning of the medical field of anesthesiology, is that chemical regulation of the CNS can have serious consequences, and the invasiveness of procedures should be carefully scrutinized. Pain relief can generally be categorized along 3 tiers of invasiveness. A first tier includes non-prescription over the counter (OTC) remedies, such as acetaminophen. A second tier includes so-called NSAIDS (nonsteroidal anti-inflammatory drugs), such as aspirin and ibuprofen, also available OTC. Prescription based remedies also include these groups, typically at increased dosage levels. Beyond this, a third group includes narcotic based remedies, such as codeine, morphine, OxyContin (oxycodone) and Percodan (percocet), to name several. In terms of treatment efficacy, there is a rather large gap between the third group and the first two, in terms of both potency and invasiveness.

Nonsteroidal Antiinflammatory Drugs NSAIDs are used primarily to treat inflammation, mild to moderate pain, and fever. Specific uses include the treatment of headaches, arthritis, sports injuries, and menstrual cramps. Aspirin (also an NSAID) is used to inhibit the clotting of blood and prevent strokes and heart attacks in individuals at high risk. NSAIDs also are included in many cold and allergy preparations.

This gap gives rise to a scenario in which a treatment from the OTC/NSAID group is in insufficient to provide relief, yet the narcotic approach is undesirable due to side effects, patient preference, or simply being overkill. Narcotics carry a greater risk of physiological addiction, and may be inappropriate for patients with a predisposition or family history of addictive patterns. Further, many states tightly regulate and track prescription history on a per-physician basis, and it may be desirable to avoid being viewed as having a propensity to over prescribe or overmedicate.

While many topical treatments (creams, rubs and the like) are commercially available, conventional topical treatments fail to develop a dosage delivery medium having the delivery focus and effectiveness as in the claimed approach. Pharmaceutical developers tend to think in narrow channels, often driven by marketability and sales concerns rather than therapeutic value. Thus, a particular medication and/or administration medium may not be pursued until a sufficiently marketable demand is established, particularly when alternative approaches may assuage the same niche. In contrast, the claimed approach provides a combination of pharmaceuticals that require a prescription synergistically combined in a transdermal delivery medium as a gel form, as opposed to less effective Over-the-Counter (OTC) medications.

Pain medications typically employ one of two administrative mediums, oral and topical. Oral administration results in bloodstream absorption of the medication, thus delivering the active medicinal components in a general manner throughout the patient. An oral dosage sufficient to deliver a desired quantity of medication to an afflicted area may be unsafe in other regards because of the general bloodstream indirection of oral administration. Due to the indirectness of oral administration, it may be difficult to direct treatment to a particular afflicted area without incurring undesirable side effects elsewhere. Intravenous administration (IV) is also frequently employed, however this simply bypasses the gastrointestinal absorption into bloodstream based delivery of the medicine.

In a particular configuration, the transdermal gel for topical application of pain relief medication on a skin surface over pain afflicted tissue areas includes a synergistic combination of medicinal components (active ingredients) for musculoskeletal pain relief, such that the active ingredients have complementary properties for pain relief directed to an afflicted tissue area and have transdermal properties for absorption into the afflicted tissue area. The gel includes a liposomal base for transdermal application of the active ingredients to the afflicted area, in which the liposomal base is for suspending the active ingredients in communication with a skin surface proximate to affected tissue areas pending transdermal absorption into the affected tissue areas. A wetting agent is added for forming gel-like properties in combination with the liposomal base and the active ingredients, the gel-like properties for maintaining the active ingredients in communication with the skin, the active ingredients present in greater quantities than that which may be safely ingested for delivery to the afflicted area.

The active ingredients are deliverable in a manner to provide a concentration at the afflicted area such that an oral administration sufficient to produce a similar concentration at the afflicted areas would be unsafe. Thus, the transdermal application and delivery medium are effective for avoiding excessive dosing of oral or intravenous administration by delivering the medication directly to the afflicted area and avoiding misdirection to other areas of the patient physiology.

Neurotransmitters of pain sensations typically stimulate receptors on a receiving neuron. Types of receptors that are responsive to Glutamate include N-methyl-D-aspartate (NMDA) and alpha-amino-3-hydroxy-5-methylisoxa-zole-4-propionic acid (AMPA) receptors. These two common types of glutamate receptors are often present at the same synapse. NMDA receptors are modulated by the spinal column, and are often associated with chronic musculoskeletal pain, in contrast to other types of messengers and neurotransmitters that are modulated elsewhere in the CNS such as by the brain.

A particular configuration employs medicinal components directed to NMDA receptors. Pain is detected by two different types of peripheral nociceptor neurons, C-fiber nociceptors with slowly conducting unmyelinated axons, and A-delta nociceptors with thinly myelinated axons. During inflammation, nociceptors become sensitized, discharge spontaneously, and produce ongoing pain. Prolonged firing of C-fiber nociceptors causes release of glutamate which acts on N-methyl-D-aspartate (NMDA) receptors in the spinal cord. Activation of NMDA receptors causes the spinal cord neuron to become more responsive to all of its inputs, resulting in central sensitization. NMDA-receptor antagonists, such as ketamine, has been shown to suppress central sensitization. NMDA-receptor activation not only increases the cell's response to pain stimuli, it also decreases neuronal sensitivity to opioid receptor agonists. The claimed transdermal gel provides focused non-narcotic medication to an afflicted area in a higher concentration than that achievable orally.

In a particular arrangement, the disclosed external pain relieving medication is suitable for topical application on the epidermis (i.e. skin) and responsive to musculoskeletal ailments by providing a delivery medium for external application to an afflicted area of a patient, in which the external area is responsive to transdermal delivery. The disclosed transdermal gel includes a plurality of medicinal components having a synergistic effect for permitting musculoskeletal movement by countering the symptoms of musculoskeletal pain and being non-narcotic for avoiding dependency. In particular configurations, the medicinal components include a pain reliever directed to blocking spinal cord receptors, such as ketamine or other NMDA antagonist or blocker. Ketamine is a favored anesthetic for emergency patients with unknown medical history because it depresses breathing and circulation less than other anesthetics. The NMDA receptor antagonist dextromethorphan is one of the more commonly used cough suppressants. Lidocaine also provides similar pain reliving properties.

The transdermal gel also includes an anti-inflammatory for mitigating swelling and lymphatic responses that hinder musculoskeletal movement, such as ketoprofen and ibuprofen. Guaifenesin also has anti-inflammatory properties due to secretion thinning properties. The gel also includes a relaxant for providing flexibility and mitigating tightness of the afflicted area. Relaxant or anti-spasmodic properties are exhibited by gabapentin and baclofen. The delivery medium defined by topical application of the gel therefore provides a greater concentration of the medicinal components to the afflicted area than a concentration deliverable by oral or intravenous mechanisms. In other words, an oral or IV arrangement providing the same level of medicinal components via bloodstream delivery would require an unsafe level of medication and likely have detrimental side effects.

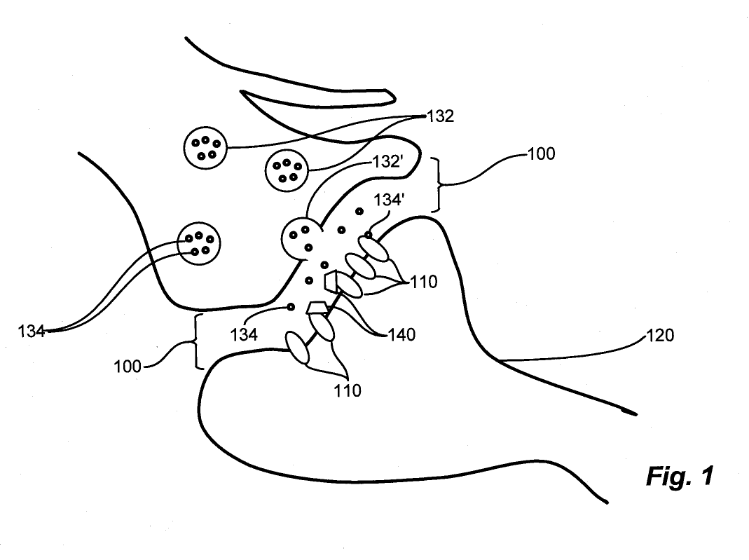

FIG. 1 is a diagram of a neural synapse 100 employing NMDA receptors 110 responsive to the present invention. Referring to FIG. 1, the synapse 100 is defined by a region, or gap, between neurons, shown in an exemplary manner as a dorsal horn neuron 120 and c-terminal 130. Synaptic vesicles 132 in the c-terminal (axon neuron) 130 contain neurotransmitters 134 that trigger a pain response. The synaptic vesicle 132′ releases neurotransmitters 134 into the synapse 100 to bind 134′ with corresponding regions of the NMDA receptor 110. NMDA receptor antagonists, or blockers 140, interfere with binding 134′ of the NMDA receptors by binding at or near receptive regions on the receptor 110 to suppress the pain stimuli.

Musculoskeletal pain typically affects the muscles, ligaments and tendons, along with the bones. Muscle tissue can be damaged with the wear and tear of daily activities. Trauma to an area, such as jerking movements, auto accidents, falls, fractures, sprains, dislocations, and direct blows to the muscle also can cause musculoskeletal pain. Other causes of pain include postural strain, repetitive movements, overuse, and prolonged immobilization.

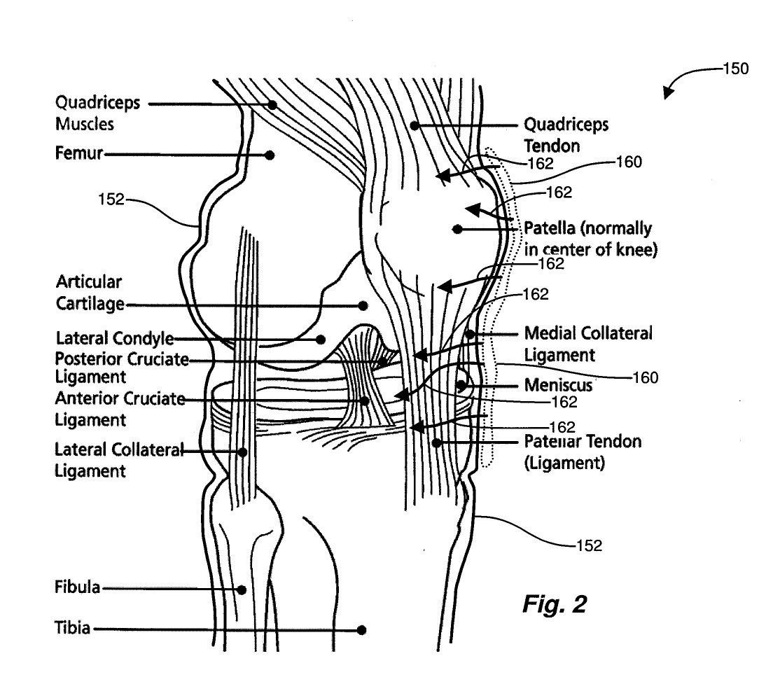

FIG. 2 is an anatomical cross section showing an epidermal region suitable for use with the transdermal gel of the present invention. The cross section 150 depicts a human knee joint and the structure of associated musculoskeletal elements responsive to the medicinal components. Application of the transdermal gel 160 on the epidermis 152 of the patient results in absorption of the medicinal components, as shown by arrows 162. Absorption by the transdermal gel provides the delivery medium for the medicinal components including the blockers 140 for suppressing the pain response.

Chronic pain can be maintained by a state of sensitization within the central nervous system that is mediated in part by the excitatory amino acids glutamate and aspartate binding to the N-methyl-D-aspartate (NMDA) receptor. A number of antagonists to the NMDA receptor are antinociceptive in animal models but are associated with significant dose-limiting side effects. Commercially available NMDA-receptor antagonists include ketamine, dextromethorphan, memantine, and amantadine. The opioids methadone, dextropropoxyphene, and ketobemidone are also antagonists at the NMDA receptor.

The NMDA receptor is known as an ionotropic receptor that allows for the transfer of electrical signals between neurons in the brain and in the spinal column. For electrical signals to pass, the NMDA receptor must be open. To remain open, an NMDA receptor must bind to glutamate and to glycine. An NMDA receptor that is bound to glycine and glutamate and has an open ion channel is called “activated.” Chemicals that deactivate the NMDA receptor are called antagonists or “blockers” because they bind to the receptor or a nearby (allosteric) site such that reception is inhibited.

In a particular configuration, the transdermal gel is a Ketamine/Ibuprofen/Lidocaine/Gabapentin/Baclofen formulation in 10%/10%/5%/6/3% concentration, respectively. A 30 gram dose is prepared as follows in Table I:

TABLE I

| | Quantity: | |
|---|---|---|
| Active Ingredient: | | |
| Ketamine | 3 | gm |
| Ibuprofen USP | 3 | gm |
| Lidocaine USP | 1.5 | gm |
| Gabapentin | 1.8 | gm |
| Baclofen HCI, USP | 0.6 | gm |
| Wetting Agent: | | |
| Ethoxy Diglycol | 3 | mL |
| Base: | | |
| Liposomal Base | 17.3 | gm |

Preparation of the transdermal gel is as follows:
1. Measure and mix dry powder ingredients in glass mortar and Pestle until uniformly mixed.
2. Slowly incorporate ethoxy diglycol into mixture #1 until creamy consistency is formed.
3. Slowly add liposomal base in geometric dilution and bring it up to desired quantity.
4. Add Thickening agent if necessary.
5. Run gel through ointment mill to further reduce particle size of active ingredients.
6. Dispense in a jar or in an ointment tube.
Such a formulation may be expected to be viable for use for 90 days.

While the system and method for pain relief using the disclosed transdermal gel has been particularly shown and described with references to embodiments thereof, it will be understood by those skilled in the art that various changes in form and details may be made therein without departing from the scope of the invention encompassed by the appended claims.

1. An external pain relieving medication responsive to musculoskeletal ailments comprising:
a delivery medium for external application to an afflicted area of a patient, the external area responsive to transdermal delivery;
a plurality of medicinal components, the medicinal components having a synergistic effect for permitting musculoskeletal movement by countering the symptoms of musculoskeletal pain and being non-narcotic for avoiding dependency, the medicinal components including:
a pain reliever directed to blocking spinal cord receptors;
an anti-inflammatory for mitigating swelling and lymphatic responses that hinder musculoskeletal movement; and
a relaxant for providing flexibility and mitigating tightness of the afflicted area;
the delivery medium providing a greater concentration of the medicinal components to the afflicted area than a concentration deliverable by oral or intravenous mechanisms.

2. The medication of claim 1 wherein the blocking is directed to spinal cord based NMDA receptors, the blocking such that the blocked impulses otherwise trigger a patient pain sensation via spinal cord receptors.

3. The medication of claim 1 wherein the pain reliever blocks NMDA receptor activation.

4. The medication of claim 3 wherein the delivery medium is a gel for delivering the medicinal components to the afflicted area via transdermal absorption.

5. A transdermal gel for topical application of pain relief medication on a skin surface over pain afflicted tissue areas comprising:
a plurality of active ingredients for pain relief, the active ingredients, the active ingredients having complementary properties for pain relief of an afflicted tissue area and having transdermal properties for absorption into the afflicted tissue area;
a liposomal base for transdermal application of the active ingredients to the afflicted area, the liposomal base for suspending the active ingredients in communication with a skin surface proximate to affected tissue areas pending transdermal absorption into the affected tissue areas; and
a wetting agent for forming gel-like properties in combination with the liposomal base and the active ingredients, the gel-like properties for maintaining the active ingredients in communication with the skin, the active ingredients present in greater quantities than that which may be safely ingested for delivery to the afflicted area.

6. The transdermal gel of claim 5 wherein the active ingredients are deliverable in a manner to provide a concentration at the afflicted area such that an oral administration sufficient to produce a similar concentration at the afflicted areas would be unsafe.

7. The transdermal gel of claim 6 wherein the transdermal application and delivery medium are for avoiding excessive dosing of oral or intravenous administration.

8. The transdermal gel of claim 7 wherein the active ingredients include:
a pain reliever directed to blocking spinal cord receptors;
an anti-inflammatory for mitigating swelling and lymphatic responses that hinder musculoskeletal movement; and
a relaxant for providing flexibility and mitigating tightness of the afflicted area.

9. The transdermal gel of claim 8 wherein the active ingredients comprise:
ketamine 10%
ketoprofen 20%
lidocaine USP 5%
gabapentin 6%
guaifenesin 10%; and
the wetting agent is ethoxy diglycol.

10. The transdermal gel of claim 8 wherein the active ingredients comprise:
ketamine 10%
ibuprofen USP 10%
lidocaine USP 5%
gabapentin 6%
baclofen HCl, USP 2%; and
the wetting agent is ethoxy diglycol.

11. A method for formulating a pain relieving gel for transdermal application of pain relief medication comprising:
mixing a predetermined quantity of active ingredients, the active ingredients having complementary properties for pain relief of afflicted tissue and having a powder form, the active ingredients occurring in greater quantities than that which may be safely ingested for delivery to the afflicted area;
adding a liposomal base, the liposomal base for transdermal application of the active ingredients to the afflicted area, the liposomal base for suspending the active ingredients in communication with a skin surface proximate to afflicted tissue areas pending transdermal absorption into the afflicted tissue areas; and
adding a wetting agent, the wetting agent for forming gel-like properties in combination with the liposomal base and the active ingredients, the gel-like properties for maintaining the active ingredients in communication with the skin.

12. The method of claim 11 wherein the active ingredients are medicinal components including a pain reliever, an anti-inflammatory, and a relaxer.

13. The method of claim 12 wherein the pain reliever suppresses proximate musculoskeletal pain generators that otherwise trigger spinal cord NDMA receptors.

14. The method of claim 13 wherein the medicinal components are non-narcotic substances having non-addictive properties.

15. The method of claim 14 wherein the medicinal components are delivered in a manner to provide a concentration at the afflicted area such that an oral administration sufficient to produce a similar concentration at the afflicted areas would be unsafe.

16. The method of claim 14 wherein the transdermal application and delivery medium avoid excessive dosing of oral or intravenous administration.

17. The method of claim 16 wherein the transdermal application delivers prescription strength levels of medication not available in over-the-counter (OTC) formulations.

18. The medication of claim 2 wherein the pain reliever is a non-narcotic NMDA antagonist having non-addictive properties, the anti-inflammatory is an NSAID for reducing inflammation and swelling that increase pain response, and the relaxant is an anti-spasmodic for controlling muscular activity.

19. The medication of claim 2 wherein the pain reliever includes ketamine and lidocaine, the anti-inflammatory includes at least one of ketoprofen or ibuprofen, and the relaxant is baclofen.

20. The transdermal gel of claim 5 wherein the active ingredients include ketamine, lidocaine, gabapentin, at least one of ketoprofen or ibuprofen, and at least one of guaifenesin or baclofen, at least one of the active ingredients in concentration greater than that available via over-the-counter formulations.
